(19) United States
(12) Patent Application Publication
(10) Pub. No.: US 2016/0038628 A1
(43) Pub. Date: Feb. 11, 2016

(54) WETNESS INDICATOR WITH PERMANENT COLORANT

(71) Applicant: The Procter & Gamble Company, Cincinnati, OH (US)

(72) Inventors: Thomas James KLOFTA, Cincinnati, OH (US); Johannson Jimmy TEE, JR., Mason, OH (US)

(21) Appl. No.: 14/819,501

(22) Filed: Aug. 6, 2015

Related U.S. Application Data
(60) Provisional application No. 62/034,355, filed on Aug. 7, 2014.

(51) Int. Cl. A61L 15/56; G01N 21/80; A61L 15/34; A61L 15/26; A61F 13/42; A61L 15/20

(52) U.S. Cl. CPC A61L 15/56; A61F 13/42; A61L 15/20; A61L 15/34; A61L 15/26; G01N 21/80; A61F 2013/422; A61F 2013/427; A61F 2013/429

(57) ABSTRACT

A wetness indicating composition is provided, comprising a liquid-activated colorant, a permanent colorant, and a binding matrix.

### FIELD OF INVENTION

Disclosed are wetness indicating compositions comprising a permanent colorant, for use in absorbent articles.

### BACKGROUND OF THE INVENTION

Many disposable absorbent articles comprise a wetness indicator. Wetness indicator compositions may comprise a colorant adapted to change in appearance, i.e., appear, disappear, change color, etc., upon contact with liquids such as urine, runny bowel movements, menses, etc., in the article. The color changing active used in many wetness indicator compositions are pH indicators such as bromocresol green or the like, which changes color from yellow to blue in the pH range of 3.8 to 5.4. Upon contact with a liquid, such as urine, the pH indicator will change colors to indicate the presence of the liquid, due to the higher pH of the urine.

However, current pH-based wetness indicators may be unreliable, having issues such as premature triggering and/or leaching, plus there are limits as to the variety of color options available. Therefore, there is a continuing need for simple wetness/fluid indicators. There is also a continuing need for ways to expand on the color options used in such wetness/fluid indicator systems, and ways to incorporate such wetness/fluid indicators into absorbent articles.

### SUMMARY OF THE INVENTION

A wetness indicating composition is provided, comprising a liquid-activated colorant, a permanent colorant, and a binding matrix.

### BRIEF DESCRIPTION OF THE DRAWINGS

FIG. 1 is a top view of an absorbent article according to an aspect of the invention.

### DETAILED DESCRIPTION OF THE INVENTION

### Definitions

“Absorbent article” refers to devices which absorb and contain body exudates and, more specifically, refers to devices which are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” means a structure typically disposed between a topsheet and backsheet of an absorbent article for absorbing and containing liquid received by the absorbent article and may comprise one or more substrates, absorbent polymer material disposed on the one or more substrates, and a thermoplastic composition on the absorbent particulate polymer material and at least a portion of the one or more substrates for immobilizing the absorbent particulate polymer material on the one or more substrates.

“Absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01).

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Fiber” and “filament” are used interchangeably.

A “nonwoven” is a manufactured sheet, web, or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants.” Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992.

“Substantially surfactant free” is used herein to describe an article component, such as a dusting layer, that contains less than 10% by weight of a surfactant or mixture thereof, less than 5% by weight of surfactant, less than 1% by weight of surfactant, no surfactant, or no more than an immaterial amount of surfactant where the surfactant may be anionic, cationic, nonionic, amphoteric or may include mixtures thereof and function to increase the wettability of the article component by reducing the contact angle of synthetic urine (as disclosed in U.S. Pat. No. 6,772,708 to Klofta) in contact with the surface of the article component (e.g., fibers of a nonwoven material or the surface of a film).

### Colorants

The wetness indicating compositions that are utilized in this invention comprise at least one liquid-activated colorant combined with at least one permanent colorant. A colorant may be a dye, an ink, a pigment, or a pH indicator. A permanent colorant is one that does not substantially change its color at any time while in the wetness indicator matrix. That is, a permanent colorant is one that is substantially the same color in the dry and wet state of the particular wetness indicator matrix, also stated as substantially the same color in an initial state and its final state. A permanent colorant may be, for example, a pigment or a dye or even a liquid-activated pH indicator colorant. While it may seem counter-intuitive that a pH indicator colorant may be defined as a permanent colorant, as they change colors as a function of the pH environment, it is possible to maintain a substantially permanent and constant color of the pH indicator colorant if the pH of the dry and wet state environments can be maintained either on the acid range or basic range of the pH indicator colorant. For example, consider the use of phloxine B (also known as D&C Red 28) as a permanent colorant. Phloxine B has a pKa of around 2.9 such that if the environmental pH is in its acid range below 2.9, the phloxine will be colorless, especially if the pH is at least one unit less than the pKa of the phloxine B. If the environmental pH is above this pKa of 2.9, the color of the phloxine pH indicator colorant is pinkish-purple, especially if the pH is a unit or more above the pKa of the phloxine B. Thus, if one can maintain the pH of both the dry state indicator and wetting fluid above phloxine's pKa of 2.9, the phloxine B will permanently remain in its basic color of pinkish-purple. This could be accomplished by formulating the phloxine B with a weak carboxylic acid based stabilizer like a fatty acid in the dry state such that the dry wetness indicator composition is pinkish-purple in color. Upon contact with urine at a pH around 6, the color remains substantially the same as pinkish-purple.

It is important also for all colorants to substantially stay in the wetness indicator matrix in which they originate, even when a fluid such as urine is introduced. It is important for the colorant to substantially remain in the wetness indicator matrix after it is wetted so the caregiver can observe the color change on the backsheet. If the colorant substantially leaches out of the wetness indicator matrix and diffuses into the core of the absorbent article, the color change is more difficult to detect by the caregiver, or in worst cases, could leach through the backsheet and stain the baby's clothing or bed sheets.

Permanent colorants can function to change the color hue of the wetness indicator composition of either its dry state or color changed state after contact with a fluid like urine. Some examples of oil soluble permanent colorants include D&C Yellow No. 11, D&C Red No. 17, D&C Red No. 21. D&C Red No. 27 (also know as acid phloxine B), D&C Violet No. 2, D&C Green No. 6, and D&C Orange No. 5. Additional permanent colorants include Pigment Red 146 (CAS#5280-68-2), Pigment Red 122 (CAS#980-26-7), Pigment Orange 16 (CAS#6505-28-8), food colorants like FD&C Blue No. 1, FD&C Red No. 40, annatto, lycopene, carmine, elderberry juice, red beet extract, and beta-carotene. These permanent colorants can not only change the color hue of the wetness indicator composition in either the dry or wet state, but they can be advantageous since many of them have a reduced solubility in hydrophilic liquids like urine. Thus, their leaching is inhibited and they possess a higher probability of remaining bound within the predominately lipophilic wetness indicator composition after being wetting with an aqueous liquid like urine. In certain instances, hydrophilic colorants which are more soluble in polar solvents like water, can also function as permanent colorants. This can occur when the hydrophilic colorant is bound within the wetness indicator composition due to strong bond formation between the colorant and another ingredient within the wetness indicator composition. For example in certain instances, a hydrophilic and anionic colorant could become permanently bound within the wetness indicator composition due to strong bond formation with a cationic binding agent or, a hydrophilic and cationic colorant could become permanently bound within the wetness indicator composition due to strong bond formation with an anionic binding agent.

A liquid-activated colorant is a colorant that changes color within the wetness indicator composition after being insulted by a fluid like urine. Simply put, the liquid-activated colorant is one unique color in the dry state and a different and unique color after being wetted with a body fluid like urine. So a liquid-activated colorant is a first color in its initial state or dry state, and a second color in its final state or wet state. For example, the wetness indicator might be yellow in the dry state and change to blue in the wet state because the liquid-activated colorant changes color as a function of pH. Thus, a liquid-activated colorant like bromocresol green could be formulated into the wetness indicator at a pH below its pKa with the inclusion of an acid. Being below its pKa within the dry wetness indicating composition, the bromocresol green will be in its yellow free acid form. After being insulted by a body fluid like urine with a higher pH around 6 which is is above the pKa of the bromocresol green, the liquid-acitvated colorant in the form of bromocresol-green will change in color to a blue-green. This liquid activated color change caused by a change in pH is called halochromism.

The color change for the liquid-activated colorant could be triggered not only by a change in pH but other components within the fluid. For example, the calcium metal ion within the urine could trigger a liquid activated color change for a liquid-activated colorant which changes color upon reacting with specific metals like the divalent calcium ion. These colorants are termed metallochromic since they change color upon binding with different metals. There are also colorants that change color due to being in different solvent environments. Thus, the colorant could be one specific color in the organic and lipophilic wetness indicator composition and change color upon being insulted by the water within the hydrophilic urine. This is termed solvatochromism.

It is important to note that the liquid activated colorant can behave as a permanent colorant if the wetness indicator composition is formulated for this purpose. For example, a solvachromic liquid-activated colorant might be yellow in a lipophilic based binding matrix like a hot melt adhesive composition and turn blue when a hydrophilic and aqueous body fluid like urine contacts the WI composition. But, the wetness indicator binding matrix could be formulated with a high concentration of hydrophilic ingredients like polyethylene glycols in order to turn the solvachromic indicator blue in color. Thus, the color of this dry state wetness indicator composition with a high concentration of hydrophilic polyethylene glycols would be blue and this blue color would be maintained after being contacted with a hydrophilic body fluid like urine. Thus, this solvachromic colorant would behave as a permanent colorant. As previously described, other liquid-activated indicator colorants like pH liquid-activated colorants can behave as either liquid-activated or permanent colorants depending on the pH of both the wetness indicator matrix and the fluid which contacts the wetness indicator composition. See example 2 below for a detailed composition.

Some representative examples of liquid-activated colorants that can be used in the practice of this invention include: malachite green, brilliant green, crystal violet, erythrosine B, methyl green, methyl violet 2D, picric acid, naphthol yellow S, quinaldine red, eosine Y, metanil yellow, m-cresol purple, thymol blue, xylenol blue, basis fuchsin, litmus, eosin B, 4-p-aminophenol(azo)benzenesulphonic acid-sodium salt, cresol red, m-cresol red, m-cresol purple, martius yellow, phloxine B, acid phloxine B, methyl yellow, methyl yellow, 2,4-dinitrophenol, orange IV, benzopurpurin 4B, bromophenol blue (free acid and conjugate base forms), congo red, methyl orange, crystal violet lactone, ethyl bis(2,4-dinitrophenyl) acetate, bromochlorophenol blue (water soluble or free acid form), ethyl orange, bromocresol green (free acid and conjugate base salt forms), chrysoidine, methyl red (acid and basic forms), alizarine red S, cochineal, chlorophenol red, bromocresol purple (free acid and conjugate base forms), alizarin, nitrazine yellow, bromothymol blue, brilliant yellow, neutral red, rosolic acid (also known as aurin), phenol red, 3-nitro phenol, orange II, phenolphthalein, 2,6-divanillyldenecyclohexanone, metacresol purple, 4-nitrophenol, o-cresolphthalein, nile blue A, thymolphthalein, aniline blue WS, alizarine yellow GG, mordant orange, tropaeolin O, orange G, methyl blue, sodium indigosulfonate, acid fuchsin, thiazol yellow G, indigo carmine, cresol red, methyl red, p-nitrophenol, and alizarin yellow R. In certain instances, it is advantageous to use the free acid form, free base form, or metal salt form, or mixtures thereof of the colorants. Also and as previously noted, many of the liquid-activated colorants can also function as a permanent colorant depending on the properties of both the wetness indicator composition and the fluid which ultimately contacts the wetness indicator composition.

Additional hydrophilic permanent colorants may include FD&C Blue No. 1, FD&C Blue No. 2, FD&C Green No. 3, FD&C Red No. 40, FD&C Red No. 4, FD&C Yellow No. 5, FD&C Yellow No. 6, C.I. Food Blue 5, and C.I. Food Red 7, D&C Yellow No. 10, D&C Yellow No. 7, D&C Yellow No. 2, D&C Yellow No. 8, D&C Orange No. 4, D&C Red No. 22, D&C Red No. 28 (also known as phloxine B), D&C Red No. 33, D&C Green No. 8, D&C Green No. 5, D&C Brown No. 1, and any combination thereof. Preferably, the colorant is soluble within the wetness indicator composition, but, as noted in certain instances, the colorant can function as intended by homogeneously suspending or dispersing it within the wetness indicator composition.

Additional suitable fluid colorants include water soluble permanent colorants like direct dyes, acid dyes, base dyes, and various solvent-soluble permanent colorants. Dispersed or suspended pigment colorants can also be employed into these wetness indicator compositions (liquid-activated formulations) although it can be more challenging to homogeneously suspend dispersed particles like pigments. Examples include, but are not limited to, C.I. Acid Yellow 73, C.I. Solvent Yellow 94, C.I. Acid Yellow 74, C.I. Solvent Orange 32, C.I. Solvent Red 42, C.I. Acid Orange 11, C.I. Solvent Red 72, C.I. Pigment Orange 39, C.I. Solvent Orange 18, C.I. Acid Red 87, C.I. Solvent Red 43, C.I. Pigment Red 90:1, C.I. Solvent Red 44, C.I. Solvent Red 45, C.I. Solvent Orange 16, C.I. Acid Red 91, C.I. Acid Red 98, C.I. Acid Red 92, C.I. Solvent Red 48, C.I. Pigment Red 174, Pigment Red 146 (C.I. No. 12485, CAS#5280-68-2)), Pigment Red 122 (CAS#980-26-7), Pigment Red 112 (CAS#6535-46-2), Pigment Red 101 (CAS#1309-37-1), Pigment Orange 34 (CAS#15793-73-4, Pigment Orange 16 (CAS#6505-28-8), Pigment Green 7 (CAS#1328-53-6), Pigment Blue 15:2 (CAS#12239-87-1), Pigment Blue 15 (CAS#147-14-8), Pigment Black 7 (CAS#1333-86-4), Pigment Red 176, Pigment Red 200, Pigment Red 254, Pigment Red 48:1, Pigment Red 48:2, Pigment Red 48:3, Pigment Red 52, Pigment Red 52:1, Pigement Red 57:1, Pigment Red 63:1, Pigment Violet 19, Pigment Violet 23, Pigment Yellow 12, Pigment Yellow 13, Pigment Yellow 14, Pigment Yellow 17, Pigment Yellow 74, Pigment Yellow 83, C.I. Acid Red 95, C.I. Solvent Red 73, C.I. Pigment Red 191, C.I. Acid Red 51, C.I. Food Red 14, C.I. Pigment Red 172, C.I. Solvent Red 140, C.I. Acid Red 93, C.I. Solvent Red 47, C.I. Acid Red 94, C.I. Solvent Red 141, C.I. Mordant Violet 25, C.I. Solvent Orange 17, C.I. Solvent Red 46, D&C Red 27 (C.I. 45410:1), D&C Orange 5 (C.I. 45370:2), and combinations thereof. More preferred permanent colorants are selected from the group consisting of D&C Red 27 (also known as Acid Phloxine B), D&C Orange 5, and combinations thereof.

Additional suitable colorants that sometimes may function as a permanent colorant and other times may function as a liquid-activated colorant may include bromopyrogallol red, bromoxylenol blue, methylene blue, monoazo dyes such as acid alizarin voliet N, monoazo pyrazoline dyes (such as acid yellow 34), diazo dyes (such as acid black 24), anthraquinone dyes (such as acid black 48), amphoteric anthraquinone dyes (such as acid blue 45), triphenylmethane dyes (such as acid fuchsin), phthalein type dyes (such as o-cresolphthalein), xanthene dyes (such as 2′7′ dichlorofluorescein eosin B), heterocyclic acridine aromatics (such as acridine orange), diphenylmethane dyes (such as auramine O), triphenylmethane dyes (such as basic fuchsin), cationic thiazine dyes (azure C), cationic anthraquinone dyes such as basic blue 47, phthalocyanine type dyes (such as strazon orange G), anthraquinone type (such as alizarin), neutral complex dyes (such as azure A eosinate), terpene type dyes (such as trans-beta-carotene), as well as combinations including at least one of the foregoing dyes.

Examples of colorants that may sometimes function as a permanent colorant and other times as a liquid-activated colorant, depending on the environment of the wetness indicator, further include, but are not limited to, organic dyes, inorganic pigments, colored macromolecules, colored nanoparticles and materials. Examples of dyes include acridine dyes, anthraquinone dyes, arylmethane dyes, azo dyes, nitro dyes, nitroso dyes, phthalocyanine dyes, quinone-imine dyes, aazin dyes, Indophenol dyes, oxazin dyes, oxazone dyes, thiazole dyes, xanthene dyes, fluorene dyes, fluorone dyes, rhodamine dyes and natural dyes like beta-carotene, annatto, cochineal, caramel color, red beet extract, beet pigments, riboflavin, anthocyanin, carotenoids, apocarotenal, and paprika. Also suitable are carmelizing ingredients used to darken the color. Examples of pigments which are most typically used as permanent colorants include cadmium pigments: cadmium yellow, cadmium red, cadmium green, cadmium orange; carbon pigments: carbon black (including vine blac, lamp black), ivory black (bone char); chromium pigments: chrome yellow and chrome green; cobalt pigments: cobalt violet, cobalt blue, cerulean blue, aureolin (cobalt yellow); copper pigments: zzurite, han purple, han blue, egyptian blue, malachite, paris green, phthalocyanine blue BN, phthalocyanine green G, verdigris, viridian; Iron oxide pigments: sanguine, caput mortuum, oxide red, red ochre, Venetian red, Prussian blue; clay earth pigments (iron oxides): yellow ochre, raw sienna, burnt sienna, raw umber, burnt umber; Lead pigments: lead white, cremnitz white, Naples yellow, red lead; Mercury pigments: vermilion; Titanium pigments: titanium yellow, titanium beige, titanium white, titanium black; ultramarine pigments: ultramarine, ultramarine green shade; zinc pigments: zinc white, zinc ferrite. Other examples include alizarin, alizarin crimson, gamboge, cochineal red, rose madder, indigo, Indian yellow, Tyrian purple, organic quinacridone, magenta, phthalo green, phthalo blue, pigment red.

The liquid-activated colorant(s) may be from about 0.01 weight percent to about 20 weight percent of the wetness indicating composition. In some embodiments, the liquid-activated colorant(s) may be from about 0.02 to about 15 wt. %, or 0.02 to about 10 wt. %, or from about 0.02 to about 2 wt. %. In some embodiments, the liquid-activated colorant may be about 0.1 weight percent of the wetness indicating composition, or may be from about 0.01 to about 5 weight percent of the wetness indicating composition. The permanent colorant may be included in the wetness indicating composition at a level from about 0.01 wt % to about 20 wt %, or any integer range between.

### Binding Agents

A binding agent may be any material which immobilizes a liquid-activated colorant, or combination of colorants, within the matrix to hinder leaching of the colorant(s) into a diaper core or other regions of an absorbent article. To optimize the contrast and vibrancy of the colors, it is much preferred to “lock” the colorant within the matrix before and after contact with a fluid like urine. The binding agents can not only hinder the leaching of the color outside of the matrix, but also aid in binding the entire wetness indicating composition to a component of the absorbent article. For example, the binder can aid in forming a strong bond between the surface of the diaper backsheet and the wetness indicating composition. There are various materials which may be suitable for use as a binding agent in a hot melt binding matrix or solvent-based binding matrix for the wetness indicating compositions of the present invention. The binding matrix is the combination of ingredients that interact with the colorants to create a safe and effective wetness indicating composition that changes color upon being insulted by a body fluid like urine. The binding matrix consists of ingredients like binding agents, tackifiers, surfactants, stabilizers, anti-oxidants, UV-stabilizers, plasticizers, rosins, hot melt adhesive components like polymers and waxes, wetting agents, solvents and other optional ingredients.

In one embodiment, possible binding agents include, but are not limited to, rosins, rosin esters, polymerized rosins, pentaerythritol rosin esters, modified styrene-acrylic polymers and their salts, styrenated terpenes, polyterpene resins, terpene phenolics, and combinations thereof. Also suitable as binders are adhesives, quaternary ammonium compounds, quaternary polymers, rubbers, latexes and latex emulsions, waxes, surfactants, polyethylene glycols, polyvinyl alcohols, and combinations thereof.

A suitable rosin mixture may be the combination of Arizona Chemical's Sylvatac RE98 and Eastman's Poly-Pale™. The Sylvatac RE-98 is a pentaerythritol rosin ester and the Poly-Pale is a polymerized rosin. Both are economical matrix ingredients, both can contribute to a darker color in the dry state, both aid in maintaining effective cohesive and adhesive forces, and their acidic nature helps preserve the colorant in its dry state color. In addition to being a suitable binding agent, rosin esters, polymerized rosins, and pentaerythritol rosin esters may also be effective solubilizers for some of the other ingredients in these formulations. Furthermore, while not wishing to be limited by theory and as noted, the acidity of some rosin esters, polymerized rosins and pentaerythritol rosin esters are believed to contribute to the stabilization of particular dyes, such as, but not limited to, pH indicators. For example, some of these rosins contain acidic carboxylate groups which aid in keeping a colorant like bromocresol green (free acid) in its acidic yellow state. When using the free acid form of bromocresol green, this acidic yellow state is the preferred color for the dry state of the wetness indicator composition before the product is used.

In some embodiments, it may be preferable for the initial dry state of the wetness indicator composition to be completely white with no sign of any coloration. This can be accomplished by using synthetic ingredients that can be synthesized to be white. This is converse to the use of rosins and polymerized rosins which are natural materials most commonly derived from trees. These natural rosins tend to be more yellow in color in the dry state and not white. But, in certain cases, the addition of white opacifiers like titanium dioxide or sodium aluminum silicate can help hide the yellow coloration from the use of natural materials. In general, the wetness indicating compositions may be substantially free of opacifiers, or opacifiers may be from about 0 to 0.5% by weight of the wetness indicating composition. The wetness indicating compositions may also be substantially free of hydrochromic ionic compounds.

The binding material may immobilize the colorant when in its initial color state. How the binding material immobilizes the colorant when in its initial color state depends upon both what the binding material and colorant are. For example, the first binding material may work by one or more forces selected from the group consisting of adhesion, hydrogen bonding, ionic, polar covalent bonding, Van der Waals forces, dipole-dipole forces, London dispersion forces and combinations thereof.

The binding agent may be employed in compositions at levels which are effective at immobilizing and stabilizing the colorant in its first state, including from about 1% to about 90%, from about 10% to about 75%, and from about 20% to about 65%, by weight of the composition. In some embodiments, the binding matrix may be from about 25 wt % to about 75 wt % of the wetness indicating composition. In some embodiments, the binding matrix may be from about 25, 30, 35, 40, 45, or 50 wt % to about 99, 95, 90, 85, 80, 75, 70, 65, 60, or 55 wt %, or any combination therein.

The binding matrix may comprise a first and second binding agent. The second binding agent may be any material which may immobilize the colorant when the colorant is in its final color state. This immobilization helps to bind the colorant within the wetness indicator composition to prevent it from leaching to other regions of the diaper such as the diaper core. It should be noted that similar to the first binding agent, the second binding agent can function not only to hinder the leaching of the colorant outside of the wetness indicator composition but the second binding agent can also aid in bonding the entire wetness indicator composition to the material of interest within the absorbent article. For example, the second binding agent may aid in bonding the wetness indicator composition to the backsheet of the diaper. There are various materials which may be suitable for use as an additional binding agent for the wetness indicating compositions of the present invention.

In one embodiment, a binding agent may be selected from, but are not limited to, the second binding agents disclosed in U.S. Pat. No. 6,904,865 to Klofta.

In one optional embodiment of the present invention, a binding agent is selected from the group consisting of quaternary ammonium salt compounds, cationic clay, polyacrylic acid polymers, organic acids, and combinations thereof. Examples of suitable quaternary ammonium compounds include, but are not limited to, dimethyl(2-ethylhexylhydrogenatedtallowalkyl) ammonium methyl sulfate, cocoalkylmethyl[ethoxylated(15)] ammonium chloride, dodecyltrimethyl ammonium chloride, hexadecyltrimethyl ammonium methyl sulfate, octadecyltrimethyl ammonium chloride, dicocoalkyldimethly ammonium chloride, di(hydrogenated tallowalkyl)dimethyl ammonium chloride, and distearyldimethyl ammonium chloride.

It should be noted that the counter anion associated with the quaternary compound, or any binding agent having one or more cationic group, is not specifically limited to chloride. Other anions can also be employed and non-limiting examples include methyl sulfate and nitrite. Similarly, any suitable counter cation, such as, but not limited to, sodium, potassium, calcium, magnesium, zinc, protons, ammonium, substituted ammonium and the like, may be associated with a binding agent having one or more anionic groups. Cationic polymers like polyethylenimines can also hinder leaching of anionic colorants. An example of a polyethylenimine (PEI) is the Lupasol™ line of PEI's from BASF.

The second binding material may immobilize the colorant when in its final color state. How the second binding material immobilizes the colorant when in its final color state depends upon the chemical composition of both the second binding material and colorant. For example, if the colorant's final color state is that of an anionic long chain molecule and the second binding material is a cationic molecule, then the bond formed may be, for example, an ionic bond, a covalent bond, or the like, or combinations of the relevant bonding forces. Another example, if the colorant's final color state is that of a cationic molecule, and the second binding material is an anionic long chain molecule, then the bond formed may be, for example, an ionic bond, covalent bond, or the like, or combinations of the relevant bonding forces.

In one embodiment of the present invention the second binding agent immobilizes the colorant when the colorant is in its final color state by one or more selected from the group consisting of covalent bonding, ionic bonding, Van der Waals, and combinations thereof.

Without wishing to be bound by theory, it is believed that when the colorant is an anion in its final color state and the second binding agent is a cation or the colorant is a cation in its final color state and the second binding agent is an anion, the second binding agent forms an ionically bonded coacervate with the colorant. For example, when the final state associated with a colorant's final color state is the pH of urine, contacting the colorant with urine will change the colorant to its final color state, i.e. an anion, and this forms an ionic bond with the second binding agent, which is a cation. The coacervate formation is due to the strong coulombic interaction between the opposite charges of the colorant and the second binding agent. The coacervate formed between the colorant and the second binding agent neutralizes the charge in both species and dramatically reduces both of their solubilities in polar solvents such as water or urine while the coacervate's solubility in the matrix remains high due to this charge neutralization and the coacervate's more lipophilic nature. Both of these effects dramatically inhibits the leaching of the colorant from the matrix. The increased lipophilicity of the coacervate leads to increased intermolecular bonding forces between the coacervate and components of the matrix. These intermolecular forces may further limit the diffusion and mobility of the colorant into an fluid environment such as water or urine.

In certain optional embodiments of the present invention, use of cationic quaternary ammonium compounds, quaternary polymers, and combinations thereof as the second binding agent may also function to darken or intensify the color change of certain colorants, especially those belonging to the sulfonephthalein class of pH indicators. Without wishing to be bound by theory, it is believed this darkening is due to several possible factors: 1) alkaline impurities within the quaternary ammonium raw material, 2) absorption shifting and absorptivity coefficient increases due to coacervate formation and/or 3) increased formation of the colorant in its final color state.

The second binding agent may be employed in compositions at levels which are effective at immobilizing the colorant in its second state, including from about 0.5% to about 20%, from about 0.5% to about 10%, and from about 0.1% to about 5%, by weight of the composition.

In some embodiments, the binding matrix is a solvent-based binding matrix, where possible binding agents include, but are not limited to, acrylic-based solvents, alcohol-based solvents, aqueous solvents, organic solvents, and combinations thereof. Examples may include acrylates/ethylhexyl acrylate copolymers; sodium acrylate/sodium acryloyldimethyl taurate copolymer; acrylates/Octyl acrylate copolymer; ammonium polyacrylate. Examples of organic solvents may include, for example, alcohols, ketones, esters, ethers, amides, and or lactones with alcohol being preferred. Organic solvents may be selected from ethanol, propanol, butanol, acetone, tetrahydrofuran, benzene, toluene and acetonitrile. Polar solvents are preferred. Methanol is preferred. Other suitable binding agents may include acrylate/acrylamide copolymers and copolymers of vinylpyrrolidone and dimethylaminopropyl methacrylamide etc. The binder could be modified or incorporated with a commercialized varnish material or other encapsulating materials.

Water-soluble resins may also be suitable, as they may act as a binder and cause the colorant to adhere to the substrate. Examples include polyamide, cellulose derivatives, an acrylic polymer or a polyol, e.g. a water soluble resin selected from the group consisting of polyvinylpyrrolidone, polyvinyl alcohol, carboxymethylcellulose, poly(2-ethyl-2-oxazoline), polymers (homopolymers and copolymers) based on acrylic acid, polymers (homopolymers and copolymers) based on methacrylic acid), and polymers (homopolymers and copolymers) based on acrylamide and any combination thereof. Some organic soluble resin binders would include those based on acrylic, alkyd, amide, epoxy, nitrocellulose, phenolic, polyester, polyurethane, and vinyl monomers, oligomers and polymers.

Other suitable ink base material as binding agents for the color-changing compositions of the invention may be a varnish base such as a nitrocellulose compound based varnish, ethyl cellulose-based varnish, polyurethane based binding systems or a phenolic-modified co-solvent type polyamide resin-based varnish. It is believed that the ink base material may help the stability of the color-changing composition. It is also believed that the ink base material may improve the adhesion of the color-changing composition to the substrate.

In general, the solvent-based binding matrix may be from about 5% to about 75% by weight of the liquid-activated formulation.

### Hot Melt Adhesives

In some embodiments, the binding agent may be a hot melt adhesive, in some embodiments, a solvent-based binding matrix. Additional components of a hot melt adhesive binding matrix may include base polymers, tackifiers, waxes, rubbers, solvents, wetting agents, and/or anti-oxidants. Examples of base polymers used in hot melt adhesives may include ethylene-vinyl acetate (EVA) copolymers like those of the Elvax brand name and marketed by DuPont Incorporated; styrenic block copolymers like those from Kraton Incorporated, ethylene/acrylic acid copolymers like the AC brand marketed by Honeywell Incorporated which includes their AC-5120 ethylene/acrylic acid, polyvinylpyrrolidone/vinyl acetate copolymers like Ashland's S-630 polyvinylpyrollidone/vinyl acetate copolymers, polypyrrolindone homopolymers like those marketed by BASF Incorporated and marketed under the trade name of Luviskol, vinyl pyrrolidone homopolymers, polyamides; kraton polymers, ethylene/acrylic acid co-polymers, ethylene-acrylate copolymers; ethylene-vinylacetate-maleic anhydride terpolymer; ethylene-acrylate-maleic anhydride terpolymer; polyolefins such as low density and high density polyethylene, atactic polypropylene, oxidized polyethylene, polybutene-1; amorphous polyolefins like amorphous atactic propylene (APP), amorphous propylene/ethylene (APE), amorphous propylene/butane (APB), amorphous propylene/hexane (APH), and amorphous propylene/ethylene/butane; polyamides; styrene block copolymers (SBC); styrene/acrylic polymers and modified styrene/acrylic polymers; polycarbonates; silicone rubbers; polypyrrole based polymers; thermoplastic elastomers like natural and synthetic polyisoprene, polybutadiene rubber, butyl rubber, chloroprene rubber, ethylene-propylene rubber, epichlorohydrin rubber, polyacrylic rubber, polyether block amides; polymers of acrylates, alkyd resins, amides, amino resins, ethylene co-terpolymer resins such as EVA, epoxy resins, fluoropolymers, hydrocarbon resins, phenols, polyesters, olefins, polyurethanes, silicones and functionalized silicones, polystyrene and polyvinyls.

Tackifiers suitable for hot melt adhesives include, without being limited to, natural resins like copals like gum copal, dammars, mastic, and sandarac; rosins and their derivatives like Eastman Chemical's Foral AX-E hydrogenated rosin; terpenes and modified terpenes; aliphatic, cycloaliphatic, and aromatic resins like C5 aliphatic resins, C9 aromatic resins, and C5/C9 aromatic/aliphatic resins, hydrogenated hydrocarbon resins and their mixtures.

Waxes suitable for hot melt adhesives include, without being limited to, mineral waxes like paraffin and microcrystalline waxes; polyethylene waxes; polyethylene glycol type waxes like those trademarked as the Carbowax brand; oxidized polyethylene waxes; polymethylene waxes, the bisstearamides like N,N′-ethylene bisstearamide trademarked as Acrawax from Lonza Incorporated, highly branched polymer waxes like Vybar™ from Baker Hughes; fatty amide waxes; natural and synthetic waxes like beeswax, soywax, carnuba, ozokerite, ceresin; waxes derived from both the Fisher-Tropsch and Ziegler-Natta processes; water soluble waxes, polyalkylene wax, polyethylene wax, and silicone waxes.

Additional additives for adhesives and hot melt adhesives may include plasticizers, like glyceryl tribenzoate, alkyl benzoates like Eastman's Benzoflex 9-88, C12-15 alkyl benzoate, C2-C22 alkyl benzoates where the alkyl group is straight or branched or mixtures thereof, alkyl citrates, phthalates, phthalate esters, paraffin oils, and polyisobutylene; UV stabilizers; biocides and antimicrobial preservatives; antioxidants, like BHT, phospites and phosphates; antistatic agents; rosins and their derivatives; pigment, particle and powder wetting agents like polyhydroxystearic acid, polyglyceryl-4 isostearate, hexyl laurate, esters like isopropyl myristate, propylene carbonate, isononyl isononanoate, glyceryl behenate/eicosadioate, trihydroxystearin, C12-15 alkyl benzoate, C2-C22 alkyl benzoates where the alkyl group is straight or branched or mixtures thereof, triethoxycaprylysilane, castor oil; and viscosity modifiers. The wetting agent can be a combination of an ester like isononyl isononanoate and a surfactant like polyhydroxystearic acid. Optionally, solvents like mineral oil, isoparaffins, alkanes like hexane, silicone fluids, esters, alcohols, polyethylene glycols, glycerin, glycols, and water can be added to reduce the viscosity of the composition or to increase the solubility of other ingredients or change other strategic properties of the wetness indicator composition.

### Additional Ingredients

Additional ingredients may include, for example, a stabilizer, a surfactant, a plasticizer(s), a structural adjunct, and/or solvents. When present, such ingredients are typically employed in the composition at levels that are effective at providing the benefits of the ingredient or ingredients, such as, for example, from about 0.001% to about 50%, from about 0.1% to about 40%, or from about 1% to about 35%, by weight of the composition. Additional additives for adhesives and hot melt adhesives may include plasticizers, like glyceryl tribenzoate, alkyl benzoates, C12-15 alkyl benzoate, C2-C22 alkyl benzoates where the alkyl group is straight or branched or mixtures thereof, alkyl citrates, phthalates, phthalate esters, paraffin oils, and polyisobutylene; UV stabilizers; biocides and antimicrobial preservatives; antioxidants, like BHT, phospites and phosphates; antistatic agents; rosins and their derivatives; pigment, particle and powder wetting agents like polyhydroxystearic acid, polyglyceryl-4 isostearate, hexyl laurate, esters like isopropyl myristate, propylene carbonate, isononyl isononanoate, glyceryl behenate/eicosadioate, trihydroxystearin, C12-15 alkyl benzoate, C2-C22 alkyl benzoates where the alkyl group is straight or branched or mixtures thereof, triethoxycaprylysilane, castor oil; and viscosity modifiers. The wetting agent can be a combination of an ester like isononyl isononanoate and a surfactant like polyhydroxystearic acid. Optionally, solvents like mineral oil, isoparaffins, alkanes like hexane, silicone fluids, esters, alcohols, polyethylene glycols, glycerin, glycols, and water can be added to reduce the viscosity of the composition or to increase the solubility of other ingredients or change other strategic properties of the wetness indicator composition. Solvents may include a liquid, gel or semi-solid material. The solvent may be water, a thixotropic material, paste, an alcohol, ethylene glycol monobutyl ether, mineral oil, esters, silicone fluids, isoparaffins, alkanes like hexane, toluene, xylenes, low molecular weight polyethylene glycols like PEG-200, glycerin, glycols, a non-flammable solvent, an adhesive material, or other organic species. Preferred non-aqueous solvents may comprise alcohols, acetates, and combinations thereof. The alcohol solvents are preferably selected from the group consisting of iso-propyl alcohol, n-propyl alcohol, ethanol, methanol, and combinations thereof. Likewise, suitable acetate solvents include, but are not limited to, isopropyl acetate, n-propyl acetate, and combinations thereof.

Other suitable solvents that may be effective include water, aqueous detergent solutions, acidic water solutions, alkaline water solutions, isopropanol, ethanol, methyl-ethyl ketone, acetone, toluene, hexane, ethyl 15 acetate, acetic acid (vinegar), cetyl alcohol (fatty alcohol), dimethicone silicone, isopropyl lanolate, myristate, palmitate, lanolin, lanolin alcohols and oils, octyl dodecanol, oleic acid (olive oil), panthenol (vitamin B-complex derivative), stearic acid and stearyl alcohol, butylene glycol and propy lene glycol, cyclomethicone (volatile silicone), glycerin, aloe, petrolatum, and so forth. Adhesives that may be useful include, for example, those based on alkyds, animal glues, casein glues, cellulose acetates, cellulose acetate butyrates, cellulose nitrates, ethyl celluloses, methyl celluloses, carboxy methyl celluloses, epoxy resins, furane resins, melamine resins, phenolic resins, unsaturated polyesters, polyethylacrylates, poly-methylmethacrylates, polystyrenes, polyvinylacetates, polyvinylalcohols, polyvinyl acetyls, polyvinyl chlorides, polyvinyl acetate chlorides, polyvinylidene copolymers, silicones, starched based vegetable glues, urethanes, acrylonitrile rubbers, polybutene rubbers, chlorinated rubbers, styrene rubbers, and so forth. Waxes such as, for example, polyolefin waxes, bees waxes, and so forth, and gels such as, for example, glycol dimethacrylate, chitosan, polyacrylates, hydroxypropylcellulose, gelatin, and so forth, may also be useful to effect the color change. Surfactants that are suitable for the present invention may include, for example, tergitol, ethoxylated alcohols, fatty alcohols, high molecular weight alcohols, ethoxylated sorbitan esters like Tween™ 40 from Croda, the ethoxylated pareth surfactants like Performathox™ 450 from New Phase Inc., esters, polymers and other natural and synthetic waxes or olefininc materials as known in the art; anionic and cationic surfactants, alkoxylated alkylates such as PEG-20 stearate, end group-capped alkoxylated alcohols, alkoxylated glyceryl and polyglyceryl alkylates such as PEG-30 glyceryl stearate, glyceryl alkylates such as glyceryl stearate, alkoxylated hydrogenated castor oil, alkoxylated lanolin and hydrogenated lanolin, alkoxylated sorbitan alkylates, sugar derived surfactants such as the alkyl glycosides and sugar esters, poloxamers, polysorbates, and sulfo succininc acid alkyl esters. Further examples include nonionic surfactants and amphoteric surfactants and any combination thereof; specific-diethylhexylsodiumsulfosuccinate, available as MONOWET MOE75 from Uniqema, the sodium dioctyl sulfosuccinate line of surfactants like Aerosol™ OT-100 from Cytec Inc. Another example is 4-1-aminoethylphenolpolyoxyethylenefattyethers, polyoxyethylene sorbitan esters, TWEEN, and polyoxyethylene fatty acid esters.

Other suitable surfactants may be neutral block copolymer surfactants, which can be selected from polyoxypropylene-polyoxyethylene block copolymer, poly [poly(ethylene oxide)-block-poly(propylene oxide)]copolymer or propylene glycol-ethylene glycol block copolymer. Suitable non-ionic surfactants include TWEEN surfactants from Croda, such as TWEEN 20 surfactant, TWEEN 40 surfactant and TWEEN 80 surfactant, and TRITON X-100 surfactant, which are available from Sigma-Aldrich, Incorporated. Other suitable neutral surfactants include polyethylene lauryl ether, polyoxyethylene nonyl phenyl ether, polyoxyethylene oleyl phenyl ether, polyoxyethylene sorbitan monolaurate, polyethylene glycol monostearate, polyethylene glycol sorbitan monolaurate, polyoxyethylenesorbitan monopalmitate, polyoxyethylenesorbitan monostearate, polyoxyethylenesorbitan monooleate, polyoxyethylenesorbitan trioleate, polypropylene glycol sorbitan monolaurate, polyoxypropylenesorbitan monopalmitate, polyoxypropylenesorbitan monostearate, polyoxypropylenesorbitan monooleate, polyoxypropylenesorbitan trioleate, polyalkyne glycol sorbitan monolaurate, polyalkyne glycol sorbitan monopalmitate, polyalkyne glycol sorbitan monostearate, polyalkyne glycol sorbitan monooleate, polyalkyne glycol sorbitan trioleate and mixtures of such neutral surfactants.

The neutral block copolymer based surfactants include FLURONIC series block copolymers, such as PLURONIC P84 or FLURON IC P85 surfactants, which are available from BASF Corporation. Super wetting surfactants like Dupont's Capstone™ line of fluorosurfactants and Siltech's Silsurf™ A008 silicone super wetter would also be suitable at lower concentrations.

Other suitable neutral block copolymer based surfactants include nonylphenol ethoxylates, linear alkyl alcohol ethoxylate, ethylene oxide-propylene oxide block copolymer, polyoxypropylene-polyoxyethylene block copolymer, polyalkylene oxide block copolymer, polyalkylene oxide block copolymer and propylene glycol-ethylene glycol block copolymer.

It may be desirable to include a stabilizer when the colorant is a pH indicator and when the absorbent article could be stored under conditions of high humidities and temperatures. The inclusion of a stabilizer is also especially important for new diaper designs where materials and/or chemicals are present that could potentially prematurely activate the color change of the colorant within the ink formulation.

In one embodiment of the present invention, the stabilizer is an acidic stabilizer. In another embodiment of the present invention, the stabilizer is a basic stabilizer. The inclusion of a stabilizer, while not wishing to be limited by theory, is believed to play a role in stabilizing the colorant against premature changes caused by exposure to humid environments and/or certain components of the diaper, by maintaining a stable pH, such as a low pH environment with an acidic stabilizer, around the colorant even when the system is exposed to high humidities and/or certain components of the diaper. This maintenance of a stable pH environment keeps the colorant, especially when the colorant is a pH indicator, in its initial dry color state. Desiccants can also stabilize the composition by trapping free water that could prematurely activate the wetness indicator composition. Examples of suitable desiccants include silica gel, bentonite clays, activated alumina, calcium sulfate, copper(II) sulfate, and magnesium sulfate.

One of the key properties of a properly functioning wetness indicator is for it to maintain its dry state color during a variety of storage and packaging conditions while still undergoing a noticeable color change in a reasonable amount of time after being contacted by urine. The colorant should also remain stable to various chemicals and materials that might be present in the diaper. Although acidic moieties present in the rosins as part of the matrix can aid in preserving the dry state color, additional stabilizer ingredients have been found to be necessary with some new diaper designs where high pH components within the diaper can cause the undesirable and premature color change activation of the colorant. To maintain the colorant in its acidic dry state color, acids of suitable strength should be added. Suitable strength is defined by the colorant and pH range where it changes color. The colorant's pKa value is especially important in assessing the characteristics of the chosen stabilizer.

For a pH indicator colorant like the sulfonephthalein class which includes bromocresol green which changes color between a pH of 3.8 and 5.4 (See “The Sigma-Aldrich Handbook of Stains, Dyes and Indicators,” by Floyd J. Green, Aldrich Chemical Co., Milwaukee, Wis.), the stabilizer should contribute suitably strong acidic moieties to keep the bromocresol green in its yellow state within the matrix. Although many strong acids like sulfuric acid and hydrochloric acid have suitably low pH's to accomplish this, their solubilities are low in these anhydrous matrices. In addition, their high acidity can chemically decompose the structures of some of the components present in the wetness composition and diaper. As noted, carboxylic acid moieties present in the matrix ingredients like rosins or polymerized rosins can also aid in maintaining the colorant in its acidic color state but carboxylic acids are typically too weak to maintain the dry yellow state of bromocresol green if it is exposed to high humidities and/or high pH components within new diaper designs. To increase the strength of the carboxylic acids, one can add electron withdrawing groups between the carboxylic acid moiety and another portion of the molecule. Although a fatty acid like stearic acid can aid in preserving the dry state color, it can be made more effective by making it a stronger acid by inserting polyoxyethylene groups between the carboxylic acid group and the alkyl chain. These types of molecules are called ether carboxylates and these acidic molecules can be effective in maintaining the dry state acid form of the pH indicator colorant like bromocresol green. In addition, the alkyl group present in these ether carboxylates increases their solubility in the wetness indicator matrix. Finally, the ether carboxylate's surfactancy can aid in increasing the kinetics for activating the color change of the wetness indicator composition after it is contacted by urine.

Other suitable stabilizers are those of the monoalkyl phosphate free acid and dialkyl phosphate free acid types. The phosphate acid moiety is a stronger acid than the carboxylic acid group and thus can be more effective in maintaining the low pH environment required to keep the pH indicator colorant in its dry acidic state. These alkyl phosphate free acids have been found to be particularly effective in preserving the dry state color of the bromocresol green colorant from premature activation as caused by high humidities or destabilizing materials and/or chemicals present in new diaper designs. Particularly effective alkyl phosphate free acids are stearyl phosphate free acid, cetyl phosphate free acid, and cetearyl phosphate free acids. Thus, the phosphate is a suitably strong acid to maintain the pH indicator colorant in its acidic dry state form, and the lipophilic alkyl moiety aids in increasing its solubility within the wetness indicator composition. In addition, the surfactant nature of the alkyl phosphate free acids can aid in speeding up the kinetics of the color change after the wetness indicator composition is contacted by urine.

Other acidic stabilizers which are particularly effective in stabilizing the wetness indicator formula to high humidities and/or destabilizing components within the diaper include, but are not limited to: organic acids, such as, but not limited to, fatty acids such as stearic acid, palmitic acid, lower molecular weight acids such as citric acid, malic acid, maleic acid, lactic acid, glycolic acid, gluconic acid, fumaric acid, oxalic acid, adipic acid, ascorbic acid, and salicylic acid; acid esters, such as, citrate esters, e.g., monostearyl citrate and monocetyl citrate, glycolate esters, lactate esters; phosphorus containing organic acids, such as, monostearyl phosphate and monocetyl phosphates; ether carboxylic acids; N-acyl sarcosinic acids; N-acyl glutamic acids; N-acyl ethylenediaminetriacetic acid; alkane sulfonic acids; alpha-olefin sulfonic acids; alpha-sulfonic acid fatty acid methyl esters; sulfate esters; inorganic acids, such as, phosphoric acid; and combinations thereof. Examples of suitable basic stabilizers include, but are not limited to: monoethanolamine; diethanolamine; triethanolamine; dipropylenetriamine; diiosopropyl amine; organic diamines, such as, but not limited to, 1,3-bis(methylamine)-cyclohexane, 1,3-pentanediamine; inorganic bases, such as, but not limited to, sodium hydroxide, magnesium hydroxide, and combinations thereof.

The stabilizer, when present is typically employed in compositions at levels which are effective at stabilizing the colorant, from about 0.001% to about 30%, from about 0.1% to about 15%, and also from about 1% to about 10%, by weight of the composition.

The present invention may include structural adjuncts, such as HLB (hydrophilic lipophilic balance) modifiers, viscosity modifiers, hardening agents, wetting agents, anti-oxidants, anti-leaching aids, and/or colorant solubilizers. Suitable ones may include polymeric thickeners such as block copolymers having polystyrene blocks on both ends of a rubber molecule, the aforementioned copolymers of ethylene and vinyl acetate (EVA), hydrogenated castor oil, polymers, metals salts of fatty acids, silicas and or derivatized silicas, organoclays such as modified and unmodified hectorites and bentonites, modified clays such as modified laponite clays, dibenylidene sorbitol, alkyl galactomannan, aluminium magnesium hydroxide stearate/oil blends and lauroyl glutamic dibutylamide. Hardeining agents may include the aforementioned waxes, C14-22 fatty alcohols, C14-22 fatty acids, C23-60 carboxylic acids, hydrogenated vegetable oils, polymers, sorbitan esters and other high molecular weight esters.

The wetting agent can be a surfactant or a mixture of surfactants. The surfactants can be non-ionic surfactants or ionic surfactants. The ionic surfactants can be either positively charged or negatively charged or have both cationic and anionic charges where such a molecular structure is termed to amphoteric. The examples of non-ionic surfactants include alkyl poly(ethylene oxide) such as copolymers of poly(ethylene oxide) and poly(propylene oxide) (commercially called Poloxamers or Poloxamines), alkyl polyglucosides such as octyl glucoside and decyl maltoside, fatty alcohols such as cetyl alcohol, oleyl alcohol, cocamide MEA and cocamide DEA. The examples of ionic surfactants include anionic (e.g., based on sulfate, sulfonate or carboxylate anions) surfactants such as s(SDS), ammonium lauryl sulfate and other alkyl sulfate salts, Sodium laureth sulfate, also known as sodium lauryl ether sulfate (SLES), Alkyl benzene sulfonate, Soaps, or fatty acid salts; and Cationic (e.g., based on quaternary ammonium cations) surfactants such as Cetyl trimethylammonium bromide (CTAB) a.k.a. hexadecyl trimethyl ammonium bromide, and other alkyltrimethylammonium salts, Cetylpyridinium chloride (CPC), Polyethoxylated tallow amine (POEA), Benzalkonium chloride (BAC), Benzethonium chloride (BZT); or Zwitterionic (amphoteric) surfactants such as Dodecyl betaine, Dodecyl dimethylamine oxide, Cocamidopropyl betaine, Coco ampho glycinate. Alternatively, the wetting agents may also be hydrophilic molecules. The hydrophilic molecules may be small molecules such as sucrose, glucose and glycerol. The hydrophilic molecules may also be polymers such as polyethylene glycol and its copolymers.

### Substrate and Absorbent Article

In one embodiment of the present invention, the wetness indicating composition of the present invention may be on and/or in a substrate. When present on a substrate, the wetness indicator formulation will typically be placed on and/or in a substrate where the substrate will be contacted by a liquid, such as water, urine, menses, blood and the like. The substrate may include, but is not limited to, a structural component, such as woven fabrics, nonwoven fabrics, films, sponges, and combinations thereof. The substrate may comprise synthetic and/or natural materials. In one embodiment of the present invention the optional substrate may be an article in its own right, such as, a continuous nonwoven fabric. In another embodiment of the present invention the substrate to which the wetness indicating composition may be applied or otherwise affixed comprises any one, or a combination of, structural components of an absorbent article, including, but not limited to, the backsheet, topsheet, fasteners, absorbent material, etc., or may be a separate element added or applied to the product. In one optional embodiment of the present invention the wetness indicating composition is applied to the absorbent article as a whole. In some embodiments, the wetness indicating composition is a single layer. Such a single layer may be applied to a substrate or structural component. In some embodiments, the single-layer formulation may be disposed between the backsheet and the absorbent core, in other embodiments, between the topsheet and the absorbent core.

The indicating material may be coated over a surface of said substrate as either a) a monochromic color scheme alone, bi-chromic, or multiple colors, b) in various shapes and sizes, c) graphics of patterns or alpha numeric symbols and words, or combinations thereof. The color transition may be from being either a) colored to uncolored, b) uncolored to colored, c) colored to different colored, or d) a combination of a) and b) and c).

The following discussion is for convenience of formulation, but is not intended to limit the type of substrate used herein.

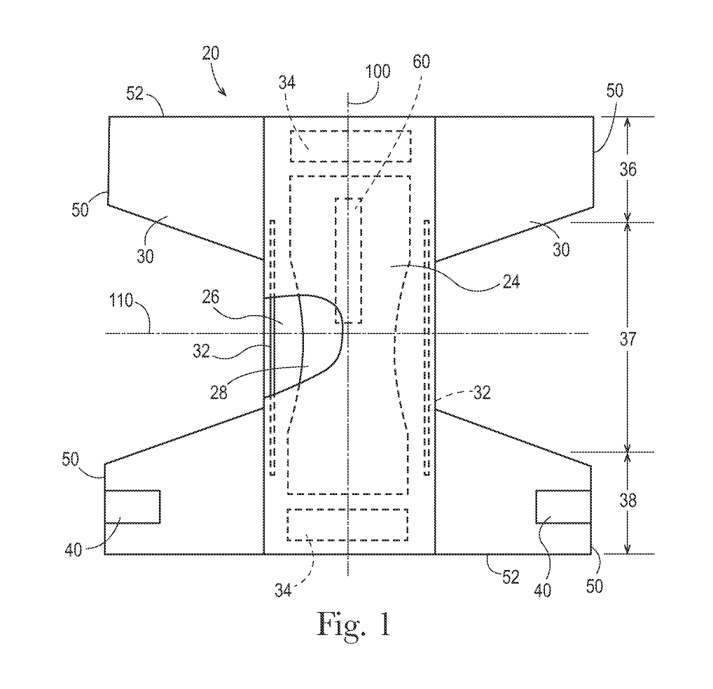

In one embodiment of the present invention the absorbent article is a disposable diaper. FIG. 1 is a plan view of a diaper 20 of the present invention in a flat-out, state with portions of the structure being cut-away to more clearly show the construction of the diaper 20. The portion of the diaper 20, which faces the wearer, is oriented towards the viewer. As shown in FIG. 1, the diaper 20 preferably comprises a liquid pervious topsheet 24; a liquid impervious backsheet 26; an absorbent core 28 that is preferably positioned between at least a portion of the topsheet 24 and the backsheet 26; side panels 30; elasticized leg cuffs 32; an elastic waist feature 34; and a fastening system generally designated 40. The diaper has a wetness indicator 60. The diaper 20 is shown in FIG. 1 to have a first waist region 36, a second waist region 38 opposed to the first waist region 36 and a crotch region 37 located between the first waist region 36 and the second waist region 38. The periphery of the diaper 20 is defined by the outer edges of the diaper 20 in which longitudinal edges 50 run generally parallel to the longitudinal centerline 100 of the diaper 20 and end edges 52 run between the longitudinal edges 50 generally parallel to the lateral centerline 110 of the diaper 20.

In one embodiment opposing sides of the article may be seamed or welded to form a pant. This allows the article to be used as a pull-on type diaper, such as a training pant. Additional illustrative, but non-limiting, information on construction, assembly, and the various components (including backsheets, dusting layers, upper and lower covering sheets, and webs) of disposable diapers may be found in U.S. Pat. No. 3,860,003 to Buell; U.S. Pat. No. 5,151,092 to Buell; U.S. Pat. No. 5,221,274 to Buell; U.S. Pat. No. 5,554,145 to Roe et al. on Sep. 10, 1996; U.S. Pat. No. 5,569,234 to Buell et al.; U.S. Pat. No. 5,580,411 to Nease et al.; U.S. Pat. No. 6,004,306 to Robles et al.; U.S. Pat. No. 5,938,648 to LaVon et al.; U.S. Pat. No. 5,865,823 to Curro; U.S. Pat. No. 5,571,096 to Dobrin et al.; U.S. Pat. No. 7,318,820 to LaVon et al.; U.S. Pat. No. 6,962,578 to LaVon; U.S. Pat. No. 7,377,914 to LaVon;

In one alternative embodiment of the present invention a portion of the absorbent article, such as part or all of the topsheet, part or all of the barrier leg cuffs and the like, may be optionally coated with a lotion, as is known in the art. Examples of suitable lotions include, but are not limited to, those described in U.S. Pat. No. 5,607,760 to Roe on; U.S. Pat. No. 5,609,587 to Roe; U.S. Pat. No. 5,635,191 to Roe et al.; U.S. Pat. No. 5,643,588 to Roe et al.; and U.S. Pat. No. 5,968,025 to Roe et al.

### EXAMPLES

The present invention is illustrated by the following examples, which are merely for the purpose of illustration and are not to be regarded as limiting the scope of the invention or the manner in which it can be practiced. Unless specifically indicated otherwise, parts and percentages are given by weight. For Example 1, the bromocresol green (free acid) is the liquid-activated colorant which is yellow in this dry state wetness indicator composition due to the inclusion of the strong acid stabilizer of Cetyl Phosphate. The permanent colorant is D&C Red #17 which is permanently red in color and gives the final wetness indicator composition a orange color since the combination of yellow from the bromocresol green (free acid) and the red from the D&C Red #17 results in a orange hue. After being wet with a body fluid like urine, the bromocresol green turns blue-green in color due its change in conjugation as a result of forming its conjugate base since the urine's pH is higher than the pKa of the bromocresol green. The combination of the blue-green from the liquid-activated bromocresol green and the red from the permanent colorant of D&C Red #17 results in purple after being wet out by a body fluid like urine.

### Example 1

| | Amt | Alternative | | |
|---|---|---|---|---|
| Ingredient | (wt %) | Name | Function | Supplier |
| Cetyl Phosphate | 2% | Hostaphat | Stabilizer | Clariant |
| | | CC-100 | | |
| Polyhydroxystea- | 1.5% | Dispersun | Wetting Agent | Innospec |
| ric Acid | | DSP-OL100 | | |
| Stearyl Alcohol | 29% | CO-1898 | Structural | P&G |
| | | | Adjunct | Chemicals |
| Bromocresol | 0.25% | | Liquid- | Curtiss |
| green | | | Activated | Labs |
| (free acid) | | | Colorant | |
| D&C Red #17 | 0.15% | Japan Red | Permanent | Sensient |
| | | 225 | Colorant | |
| Ethoxylated (20) | 2.1% | Tween 20 | Surfactant | Croda |
| Sorbitan Ester | | | | |
| Microcrystalline | 22.0%% | Microwax | Hot Melt | Sonneborn |
| Wax | | W-835 | Adhesive | |
| | | | Component | |
| Isononyl | 1.1% | Ester | Wetting Agent | Alzo |
| isononanoate | | Wetting | | |
| | | Agent | | |
| Steareth-20 | 41.9%% | Brij S20 | Surfactant | Croda |

For Example #1, mix and heat to 80° C. the polyhydroxystearic acid, Tween 20, microwax W835, isononyl isononanoate, bromocresol green (free acid), and the D&C Red #17 until homogeneous (call this mixture A). Separately, mix and heat the Steareth-20, Cetyl Phosphate, and Stearyl Alcohol to 60° C. and mix until homogeneous (call this mixture B). Slowly add the molten mixture A to the molten mixture B while heating to 80° C. and mix until homogeneous. Prepare films at 80° C. with a draw-down wire and allow the films to cool before testing.

### Example 2

In Example 2, the liquid activated bromophenol blue is yellow in color within the dry state wetness indicator composition due to the inclusion of the ethylene acrylic acid which acidifies the bromophenol blue into its yellow acid state. Depending on the pH of the wetness indicator compositon and the fluid which ultimately contacts the wetness indicator composition, the Phloxine B can function as either liquid-activated colorant or a permanent colorant. In this Example 2, the Phloxine B functions as a permanent colorant since the Ethylene Acrylic Acid stabilizer is not a strong acid to form the colorless free acid of the Phloxine B. Instead, the pinkish-purple conjugate base of Phloxine B is formed and this combination of pinkish-purple from the Phloxine B and the yellow from the Bromophenol Blue (free acid) creates an orange hue within the dry state wetness indicator. Upon wetting with a body fluid like urine, the pinkish-purple from the permanent colorant of Phloxine B is maintained while the rise in pH from the urine causes the blue conjugate base of bromophenol blue to be formed. This combination of blue from the bromophenol blue and the pinkish-purple from the Phloxine B gives a final color of purple after wetting with a body fluid like urine.

| | Amt | Alternative | | |
|---|---|---|---|---|
| Ingredient | (wt %) | Name | Function | Supplier |
| Ethylene Vinyl | 15.2%% | Elvax 40W | Binder/ | DuPont |
| Acetate | | | Binding | |
| | | | Matrix | |
| Bromophenol | 0.22% | | Liquid- | Curtiss |
| Blue | | | Activated | Labs |
| (free acid) | | | Colorant | |
| Phloxine B | 0.08% | CAS | Permanent | TCL |
| | | #18472-87-2 | Colorant | |
| Ethylene Acrylic | 44.0% | A-C 5120 | Binder/ | Honeywell. |
| Acid Co-polymer | | | Binding | |
| | | | Matrix | |
| Performathox 480 | 24% | C20-C40 | Surfactant | Baker- |
| | | Pareth-3 | | Hughes |
| Performathox 450 | 14% | C20-C40 | Surfactant | Baker- |
| | | Pareth-3 | | Hughes |
| Irganox 1010 | 1.0% | | Anti- | BASF |
| | | | Oxidant | |
| Isononyl | 1.5% | Ester | Wetting | Alzo |
| isononanoate | | Wetting | Agent | |
| | | Agent | | |

For Example #2, mix and heat to 120° C. the Ethylene Vinyl Acetate and the Ethylene Acrylic Acid and the Performathox 450 and the Perfomathox 480 and the Irganox 1010 until homogenious. While maintaining mixing and heating at 120° C., add the bromophenol blue (free acid) and the Phloxine B and the isononyl isononanoate until everything is dissolved at 120° C. Maintain heating to carefully and safely prepare films with a draw-down wire or allow the mixture to solidify at room temperature for future use.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A wetness indicating composition comprising: (A) a liquid-activated colorant; (B) a permanent colorant; and (C) a binding matrix.

2. The wetness indicating composition of claim 1, wherein the permanent colorant is selected from the group consisting of methylene blue, D&C Yellow No. 11, D&C Red No. 17, D&C Red No. 21. D&C Red No. 27 (Acid Phloxine B) D&C Red No. 28 (Phloxine B), D&C Violet No. 2, D&C Green No. 6, D&C Orange No. 5, Pigment Red 146, Pigment Red 122, Pigment Orange 16, FD&C Blue No. 1, FD&C Blue No. 2, FD&C Green No. 3, FD&C Red No. 3, FD&C Red No. 40, FD&C Yellow No. 5, FD&C Yellow No. 6, Quinline Yellow, Carmoisine, Ponceau 4R, Patent Blue V, Green S, caramel coloring, chlorophyllin, elderberry juice, lycopene, carmine, paprika, turmeric, annatto, metal phthalocyanine type colorants, red beet extract, beta-carotene, and combinations thereof.

3. The wetness indicating composition of claim 1, wherein the permanent colorant is organic.

4. The wetness indicating composition of claim 1, wherein the permanent colorant is inorganic.

5. The wetness indicating composition of claim 1, further comprising permanent colorant pigments suspended in the composition.

6. The wetness indicating composition of claim 1, wherein the permanent colorant is a pH indicator colorant.

7. The wetness indicating composition of claim 1, wherein the permanent colorant is water-soluble.

8. The wetness indicating composition of claim 1, wherein the permanent colorant is oil-soluble.

9. The wetness indicating composition of claim 1, wherein the binding matrix is a hot melt binding matrix.

10. The wetness indicating composition of claim 1, wherein the binding matrix is a solvent-based binding matrix.

11. The wetness indicating composition of claim 1, further comprising one or more selected from the group consisting of a stabilizer, surfactant, and a structural adjunct.

12. The wetness indicating composition of claim 11, wherein said stabilizer is selected from the group consisting of monostearyl phosphate, monocetyl phosphate, citrate esters, alcohol ethoxycarboxylates, glycolate esters, lactate esters, fatty acids, ether carboxylic acids, fatty acid methyl esters, sulfate esters, fruit acids like citric acid and malic acid, inorganic acids like sulfuric acid, monoethanolamine, diethanolamine, triethanolamine, dipropyllenetriamine, diiosopropyl amine, 1,3-bis(methylamine)-cyclohexane, 1,3-Pentanediamine, sodium hydroxide, magnesium hydroxide, and combinations thereof.

13. The wetness indicating composition of claim 11, wherein said structural adjunct is selected from the group consisting of HLB modifiers, viscosity modifiers, hardening agents, and combinations thereof.

14. An absorbent article comprising the wetness indicating composition of claim 1, wherein said wetness indicating composition is affixed to a structural component of the absorbent article.

15. An absorbent article comprising the wetness indicating composition of claim 1, wherein the article comprises a backsheet, a topsheet, an absorbent core disposed between the backsheet and the topsheet, wherein the wetness indicating composition is a single layer and disposed between the backsheet and the absorbent core.

16. An absorbent article comprising the wetness indicating composition of claim 1, wherein the article comprises a backsheet, a topsheet, an absorbent core disposed between the backsheet and the topsheet, wherein the wetness indicating composition is a single layer and disposed between the topsheet and the absorbent core.

17. The wetness indicating composition of claim 1, comprising a liquid activated colorant selected from the group consisting of malachite green, malachite green hydrochloride, brilliant green, crystal violet, crystal violet lactone, erythrosine B, methyl violet 2B, methyl violet 10B, quinaldine red, m-cresol purple, thymol blue, xylenol blue, basic fuchsin, 4-p-aminophenol(azo)benzenesulphonic acid-sodium salt, litmus, cresol red, methyl yellow, 2,4-dinitrophenol, orange IV, benzopurpurin 4B, bromophenol blue, congo red, methyl orange, bromochlorophenol blue, ethyl orange, flourocene WS, bromocresol green, chrysoidine, methyl red, alizarine red S, cochineal, curcumin, esculin, anthocyanin, logwood, chlorophenol red, bromocresol purple (both free acid and conjugate salt base forms), alizarin, nitrazine yellow, bromothymol blue, brilliant yellow, neutral red, alizarin yellow R, rosolic acid, phenol red, 3-nitro phenol, orange II, phenolphthalein, thymol blue, 2,6-divanillyldenecyclohexanone, metacresol purple, 4-nitrophenol, o-cresolphthalein, nile blue A, thymolphthalein, aniline blue, chrome orange GS, alizarine yellow GG, mordant orange, tropaeolin O, methyl blue, alizarin, sodium indigosulfonate, acid fuchsin, thiazole yellow G, indigo carmine, D&C Red No. 27 (Acid Phloxine B), D&C Red No. 28 (Phloxine B), bromopyrogallol red, bromoxylenol blue, methylene blue, acid alizarin violet N, basic blue 47, ethyl bis(2,4-dinitrophenyl) acetate, metal titration indicators, pyrogallol red, alloxan tetrahydrate purum, and any combination thereof.

18. The wetness indicating composition of claim 1, comprising two or more liquid-activated colorants.

19. The wetness indicating composition of claim 1, further comprising at least one additional permanent colorant.
